(19) United States
(12) Patent Application Publication
(10) Pub. No.: US 2004/0132119 A1
(43) Pub. Date: Jul. 8, 2004

(54) Method for detecting gynecological cancers

(75) Inventor: Steven S. Witkin, New York, NY (US)

Correspondence Address: HOFFMANN & BARON, LLP, 6900 JERICHO TURNPIKE, SYOSSET, NY, 11791, US

(73) Assignee: Cornell Research Foundation, Inc.

(21) Appl. No.: 10/686,044

(22) Filed: Oct. 15, 2003

Related U.S. Application Data
(60) Provisional application No. 60/419,646, filed on Oct. 16, 2002.

(51) Int. Cl. G01N 33/574

(52) U.S. Cl. 435/7.23

(57) ABSTRACT

The invention provides a method for detecting gynecological cancers, such as endometrial cancer, ovarian cancer, cervical cancer, vulvar cancer, uterine cancer or fallopian tube cancer, by determining the presence of extracellular heat shock proteins. The invention also provides kits useful for detecting gynecological cancers.

This application asserts the priority of U.S. provisional application serial No. 60/419,646 filed Oct. 16, 2002, the specification of which is hereby incorporated by reference in its entirety.

The invention described in this application was made with funds from the National Institutes of Health, Grant Number R03 CA89700. The United States government has certain rights in the invention.

### BACKGROUND OF THE INVENTION

The heat shock proteins (hsp) are a highly conserved family of proteins present in every organism, including humans. Under physiologic conditions, these proteins act as chaperones by facilitating protein transport and polypeptide assembly in a cell.

However, when cells are exposed to stress, such as a viral or bacterial infection, exposure to toxic molecules or malignant transformation, the synthesis of heat shock proteins is elicited. Under these adverse conditions, the hsp can aid cell survival by preventing protein denaturation, incorrect aggregation and facilitate the removal of abnormal proteins.

Several members of the heat shock protein are present in increased concentration in malignantly transformed cells, including transformed gynecological cells. Therefore, intracellular hsp can be used as a marker for cancer.

For example, synthesis of the 90 kDa heat shock protein (hsp90) has been reported to be upregulated in human leukemia and breast, endometrial, lung and melanoma cencer cells (Yufu et al., Leuk. Res., 1992, 16:597-605; Lebeau et al., Oncogene, 1991, 6:1125-1132; Nambu et al., Cancer, 1996, 77:330-338; Ferrarini et al., Int. J. Cancer, 1992, 51:613-619). In addition, the 70 kDa heat shock protein (hsp70) is reported to be present in increased levels in leukemia (Yufu et al., Leuk. Res., 1992, 16:597-605), breast cancer (Ciocca et al., Int. J. Cancer, 1993, 85:570-574) and lung carcinoma cells (Yufu et al., Leuk. Res., 1992, 16:597-605). Furthermore, increased expression of the 27 kDa heat shock protein (hsp27) has been identified in human ovarian (Langdon et al., Clin. Cancer Res., 1995, 1:1603-1609) and mammary tumors (Love et al., Brit. J. Cancer, 1994, 69:743-748; Lemieux et al., Invas. Metastas., 1997, 17:113-123).

The detection of intracellular proteins, however, usually requires an invasive procedure. For example, determining the presence of intracellular hsp in malignantly transformed cells generally requires removing tissue to access the cells. Thus, the detection of cancer typically requires a biopsy, unless the tissue is readily accessible.

Invasive procedures, such as biopsies, have the disadvantage of being expensive and uncomfortable to undergo. Therefore, such procedures are usually not performed routinely. As a result, most women who are diagnosed with gynecological cancer, such as ovarian cancer, are diagnosed when their disease is at an advanced stage.

Accordingly, a simple non-invasive procedure for detecting gynecological cancers would be beneficial. It would be especially beneficial if such a procedure could be performed during one's annual visit to the doctor. As such, the cancer can be diagnosed in its early stage.

Thus, there is a need for a non-invasive procedure for the detection of gynecological cancers. Such an assay would be beneficial for promoting the early detection of these cancers.

### SUMMARY OF THE INVENTION

The above needs have been met by the present invention by providing a method for detecting gynecological cancer in a human. The method comprises obtaining extracellular gynecological fluid and determining the presence of extracellular heat shock proteins (hsp) in the fluid. The presence of extracellular hsp in the fluid indicates a gynecological cancer in the human.

In another embodiment, the invention provides a kit for detecting gynecological cancer. The kit comprises an object capable of removing extracellular gynecological fluid and an antibody specific for hsp.

### BRIEF DESCRIPTION OF THE FIGURES

FIG. 1. Detection of hsp27-cytochrome C complexes in women with gynecologic cancers. Cell-free cervical supernatants were diluted 1:10 and assayed for the presence of hsp27-cytochrome C complexes by ELISA. The dashed horizontal line indicates the mean plus two standard deviations of the values for the benign group.

### DETAILED DESCRIPTION OF THE INVENTION

The present invention is based on the discovery by the inventor that extracellular heat shock proteins (hsp) are present in humans, especially women, with gynecological cancers.

In one embodiment, the invention relates to a method for detecting gynecological cancer in a human. The gynecological cancer can by any cancer which affects one or more reproductive organ(s) and/or reproductive tissue(s) of a human, especially women. Examples of such organs and tissues include endometrium, ovaries, cervix, vulva, uterus and fallopian tube.

The first step in the method is obtaining extracellular gynecological fluid. Extracellular gynecological fluid is fluid from the gynecological region, such as a region containing any of the reproductive organs(s) and/or tissues described above. The extracellular fluid may comprise cells, but not the fluid inside the cells.

The fluid can be obtained by any method known to those skilled in the art. Typically, the fluid is obtained by non-invasive methods.

The fluid can be obtained using any object capable of removing extracellular gynecological fluid. Examples of suitable objects include those based on the principles of absorption and aspiration.

Any object suitable for absorbing gynecological fluids can be employed. Examples of suitable objects include an absorbent swab or sponge. The absorbent object can be an made from any material suitable for absorbing extracellular gynecological fluids. Such materials include natural and synthetic fibers. Examples of natural fibers include cotton, wool and silk. Examples of synthetic fibers include nylon and rayon. For instance, gynecological fluid can be collected by inserting a cotton swab into the cervix of a human, and then removing it.

Other suitable objects for removing extracellular gynecological fluids include aspirators. Aspirators generally utilize a vacuum for removing fluids. Aspirators are well known to those skill in the art, and include, for example, a syringe and a pipet.

The second step in the method is determining the presence of extracellular hsp in the fluid. Extracellular hsp as used herein means that the hsp is not associated with cells, including gynecological cancer cells. For example, extracellular hsp is not bound to, and not present inside, the cells (i.e., intracellular).

As stated above, the fluid can contain cells. However, it is not necessary to remove the cells present in the fluid, since the hsp detected in the methods of the invention is extracellular hsp. For the purpose of this specification, when reference is made to hsp, it means extracellular hsp unless otherwise indicated.

The hsp detected in the invention can be any hsp found in gynecological fluid. Some examples include hsp27, hsp70, hsp90 and combinations thereof. The hsp may, for example, be free hsp or hsp complexed to a molecule, usually a protein. The protein is typically a protein involved in apoptosis.

Examples of proteins involved in apoptosis include cytochrome C, APAF-1, caspases, and combinations thereof. One or more of these apoptotic proteins can bind to one of more hsp to form an hsp-apoptotic protein complex.

The presence of hsp in the fluid can be determined by any method known to those skilled in the art. Any molecule which binds specifically to hsp can be used. Some examples include the apoptotic proteins described above. Usually, an antibody which binds hsp will be employed.

If the antibody cannot be commercially obtained, methods to produce antibodies are known to those skilled in the art. For example, polyclonal antibodies can be isolated from mammals that have been inoculated with the protein or a functional analog in accordance with methods known in the art. Briefly, polyclonal antibodies may be produced by injecting a host mammal, such as a rabbit, mouse, rat, or goat, with the protein or a fragment thereof capable of producing antibodies. Sera from the mammal are extracted and screened to obtain polyclonal antibodies that are specific to the protein or fragment.

The antibodies are preferably monoclonal. Monoclonal antibodies can be produced by methods known in the art. These methods include the immunological method described by Kohler and Milstein, Nature 256:495-497 (1975) and by Campbell, “Monoclonal antibody technology, the production and characterization of rodent and human hybridomas” in Burdon et al., eds, Laboratory Techniques in Biochemistry and Molecular Biology, Vol. 13, Elsevier Science Publishers, Amsterdam (1985); as well as the recombinant DNA method described by Huse et al., Science 246:1275-1281 (1989).

The antibody can also be a fragment of an antibody. Examples of a fragment of an antibody include Fab and Fc fragments. Methods to produce antibody fragments are known to those skilled in the art.

The antibody which binds to hsp can be directly labeled. Methods to label an antibody are known to those skilled in the art. In general, the antibody can be modified by attachment of a detectable label moiety to the antibody, or a detectable antibody can be manufactured with a detectable label moiety incorporated therein.

The detectable label moiety can be any detectable moiety, many of which are known in the art, including radioactive atoms, electron dense atoms, enzymes, chromogens and colored compounds, fluorogens and fluorescent compounds, members of specific binding pairs, and the like.

Methods for labeling antibodies have been described, for example, by Hunter W M and Greenwood F C, Nature 144:495-496 (1962) and by David et al., Biochemistry 13:1014-1021 (1974). Additional methods for labeling antibodies have been described in U.S. Pat. Nos. 3,940,475 and 3,645,090.

The label moiety may be radioactive. Some examples of useful radioactive labels include ³²P, ¹²⁵I, ¹³¹I, and ³H. Use of radioactive labels have been described in U.K. patent document No. 2,034,323, U.S. Pat. Nos. 4,358,535, and 4,302,204, each incorporated herein by reference.

Some examples of non-radioactive labels include enzymes, chromogens, atoms and molecules detectable by electron microscopy, and metal ions detectable by their magnetic properties.

Some useful enzymatic labels include enzymes that cause a detectable change in a substrate. Some useful enzymes (and their substrates) include, for example, horseradish peroxidase (pyrogallol and o-phenylenediamine), beta-galactosidase (fluorescein beta-D-galactopyranoside), and alkaline phosphatase (5-bromo-4-chloro-3-indolyl phosphate/nitro blue tetrazolium). The use of enzymatic labels has been described, for example, in U.K. 2,019,404, EP 63,879, and by Rotman, Proc Natl Acad Sci USA 47:1981-91 (1961).

The labels can be detected by any method known to those in the art. For example, if the label is radioactive, the radioactivity can be detected with a beta counter or a gamma counter, depending on the type of radioactivity emitted. If the label is calorimetric, for example, photometric instruments can be used for analysis. Examples of photometric instruments include a spectrophotometer and an ELISA plate reader.

Examples of methods which utilize labeled antibodies include immunoassays such as, an ELISA (Current Protocols in Immunology, Wiley Intersciences, New York, 1999) and a standard blot assay. In a preferred embodiment, the presence of extracellular hsp in the fluid is determined by ELISA.

These formats are normally based on incubating an antibody with the fluid suspected of containing hsp and detecting the presence of complex formed between the antibody and the hsp. The hsp is preferably immobilized prior to detection.

Immobilization may be accomplished by, for example, binding the hsp to immobilized antibodies or to an immobilized protein which the hsp is capable of binding to.

These immunoassays may involve one step, two steps, or more steps. In a one-step assay, the hsp, if it is present in the extracellular gynecological fluid, is immobilized, as described above, and incubated with a labeled antibody. The labeled antibody binds to the immobilized hsp. After washing to remove unbound molecules, the fluid is assayed for the presence of the label.

In a two-step assay, immobilized hsp is incubated with an unlabeled first antibody. The target hsp-antibody complex, if present, is then bound to a second, labeled antibody that is specific for the unlabeled antibody. The sample is washed and assayed for the presence of the label, as described herein.

The immunometric assays described above include simultaneous sandwich, forward sandwich, and reverse sandwich immunoassays. These terms are well known to those skilled in the art. These and other immunoassays are described by David et al., U.S. Pat. No. 4,376,110.

In general, it is desirable to provide incubation conditions sufficient to cause binding of as much hsp present in the fluid as possible. The specific concentration of agents in the immunoassay, the temperature and time of incubation, as well as other such assay conditions, can be varied, depending upon various factors including the concentration of hsp in the fluid, etc. Those skilled in the art will be able to determine operative and optimal assay conditions for each determination by employing routine experimentation.

Determining the presence of hsp may be qualitative. For example, the presence of hsp may be indicated by means of an assay that leads to a change in a detectable signal, such as a change in color or the emission of radioactivity, as compared to that of a control sample. Such a change indicates the presence of hsp, thereby indicating the presence of a gynecological cancer in the human.

The above method for determining the presence of hsp may further comprise determining the amount of hsp in the fluid relative to the amount of hsp in a control sample. The quantitative assays for determining this amount may, for example, use known quantities (i.e., standards) of hsp.

These standards may be used to generate a standard curve that relates a concentration of hsp to the quantity of a detectable signal. The detectable signal can be, for example, the quantity of light emitted or absorbed (e.g., optical density) or quantity of radioactivity emitted (e.g., radioactive counts per minute).

For example, a graph of known concentrations of hsp versus optical density or radioactive counts may be used to calculate the amount (e.g., concentration) of hsp in the fluid. The amount of hsp detected in a fluid using a quantitative assay is typically compared to the amount of hsp in a control sample (i.e., background amount).

It is not, however, necessary to generate a standard curve or to calculate the amount of hsp in the fluid. Alternatively, the quantity of the detectable signal (e.g., light absorbed or emitted, or radioactivity emitted) from the fluid compared to that of a control sample (i.e., background signal) may be used as a measure of the amount of hsp in the fluid relative to the control sample. The quantity of detectable signal is indicative of the amount of hsp present in the fluid since an increase in optical density or radioactive counts correlate with an increase in the concentration of hsp. Accordingly, the quantity of detectable signal may be used as a measure of the amount of hsp in the fluid.

A control sample is typically extracellular gynecological fluid obtained from a human with a benign gynecological cancer. It is not, however, necessary to determine the background amount or the quantity of background signal (e.g., control sample) each time an assay is conducted. It is well known in the art to compare the amount of hsp or the quantity of detectable signal obtained as a measure of the amount of hsp in the gynecological fluid to that of a previously determined background amount or background signal.

An amount of hsp significantly elevated over that of the control indicates the presence of hsp in the fluid and is indicative of a gynecological cancer in the human. (It is understood that, as used herein, the amount of hsp may be indicated by the quantity of the detectable signal.)

If the amount of hsp in the control is a mean value, and the standard deviation of the mean value is known, or can be calculated, an amount is considered to be significantly greater if the amount is at least two standard deviations greater than the mean value of the control.

If the standard deviation is not known, and cannot be calculated, an amount is significantly greater if the amount is at least about 25%, preferably at least about 50%, more preferably at least about 75%, and most preferably at least about 100% greater than that of the control.

If no hsp can de detected in the control, an amount of hsp is significant if it can de detected in the fluid.

In another embodiment, the invention further includes a kit for detecting gynecological cancer in accordance with the methods of the present invention. The kit comprises an object capable of removing extracellular gynecological fluid, and an antibody specific for hsp. Suitable objects and antibodies include those described above. Optionally, at least one of the following can also be added: a label, buffers, standards containing hsp, materials for developing colorimetric labels, materials for stopping colorimetric reactions, etc.

### EXAMPLES

### Example 1

### Materials and Methods

Diagnosis of endometrial cancer was by ultrasound, endometrial biopsy and hysteroscopy. Ovarian cancer was identified histologically from surgical specimens. All tumors were defined according to FIGO classification. Benign diagnoses, cysts, polyps, myomas, uterine fibroids or hyperplastic endometrium, were confirmed by histological diagnosis. The median (range) ages of subjects were 55 (37-80) years for ovarian cancer, 63 (39-97) years for endometrial cancer and 51 (22-97) years for benign diagnoses. Only the age difference between endometrial cancer and benign diagnoses was significant (p<0.01). Almost all subjects were white: 89.4% with ovarian cancer, 88.5% with endometrial cancer and 84.9% with benign diagnoses.

Endocervical specimens were collected during a speculum internal examination. In cases where the cervix had been surgically removed specimens were taken from the posterior vagina. A cotton swab was inserted, rotated for 10 seconds and then removed and vigorously shaken into a tube containing 3 ml sterile phosphate-buffered saline (PBS). The swab was removed and the specimen transported to the laboratory within 1-4 hours. Specimens were centrifuged at 10,000g for 10 min and the supernatants collected and stored in aliquots at −80° C. until tested. All specimens were negative for visible blood contamination, and only 1 sample was obtained from each subject.

Hsp27 detection. Horse heart cytochrome C (Sigma, St. Louis, Mo.) was diluted to 50 μg/ml in carbonate buffer pH 9.8 and 0.1 ml added to wells of a microtiter plate. After an overnight incubation at 4° C. the sells were washed three times with PBS containing 0.05% Tween 20 detergent (PBS-Tween). Cervical supernatants were diluted 1:10 and added to duplicate wells. After 60 minutes in a 37° C. water bath the wells were washed with PBS-Tween and a mouse monoclonal antibody to hsp27 (StressGen, Victoria, BC) diluted 1:200 in PBS-Tween was added. After an additional 60 minutes in the 37° C. water bath the wells were again washed with PBS-Tween and next incubated for 60 minutes at 37° C. with a 1:500 dilution of alkaline phosphatase (AP)- conjugated antibody to mouse IgG (Kirkegaard & Perry, Gaitherburg, Md.). After additional washes with PBS-Tween the AP substrate, p-nitrophenylphosphate, in 10% diethanolamine buffer was added to the wells. After 20-30 minutes at room temperature the optical densities in the wells were determined at 405 nm using a microtiter plate reader. The optical densities were converted to ng hsp27/ml by reference to a standard curve generated with each assay and utilizing purified recombinant hsp27 (StressGen). The lower limit of sensitivity of the assay was 0.15 ng/ml.

Hsp27-cytochrome C detection. Microtiter plates precoated with streptavidin (Reacti-Bind, Pierce, Rockford, Ill.) were incubated with biotin-labeled goat anti-mouse IgG (Kirkergaard & Perry) diluted 1:200 in PBS-Tween. After 60 minutes at room temperature on a shaker the wells were washed in PBS-Tween and mouse monoclonal antibody to cytochrome c (R&D Systems, Minneapolis, Minn.) diluted 1:200 in PBS-Tween was added to the wells for a 60 minute incubation in a 37° C. water bath. Following washes with PBS-Tween 0.1 ml of a 1:10 dilution of cervical supernatant was added to duplicate wells. Following a 60 minute, 37° C. incubation, the wells were washed and incubated with rabbit IgG antibody to hsp27 (StressGen). After an additional 60 minutes at 37° C. the wells were washed and incubated with AP-conjugated goat anti-rabbit IgG (Company). Following a final 60 minute 37° C. incubation the wells were washed, incubated with PNPP as above and optical densities determined at 405 nm.

All assays were preformed without knowledge of the clinical diagnosis.

Statistics. Differences between patient groups in the prevalence of hsp27 or hsp27-cytochrome c complexes were evaluated by Fisher's exact test. The relation between hsp27 and hsp27- cytochrome c complexes was analyzed by Spearman's rank correlation test. Differences in age between subject groups were analyzed by the Tukey-Kramer multiple comparison test. Ap value <0.05 was considered significant.

### Example 2

### Detection of Hsp27

Since hsp27 has a high affinity for cytochrome C, an ELISA assay was developed for hsp27 quantitation in cervical specimens using cytochrome C bound to wells of a microtiter plate. The results are shown in Table 1. About ⅓ of women with ovarian or endometrial cancer, as opposed to 1/10 women with benign diagnosis, were positive for cervical hsp27(p<0.003). Among the hsp27-positive specimens, the hsp27 concentrations were higher in the cancer patients than in the benign controls.

TABLE 1
Detection of hsp27 in cervical specimens
from women with gynecologic cancers

| | No. | | Concentration |
|---|---|---|---|
| Diagnosis | women | No. hsp27-positive | (ng/ml) |
| Endometrial cancer | 52 | 18 (34.6%)^{a} | 0.2-8.7 |
| Ovarian cancer | 47 | 18 (38.3%)^{b} | 0.2-10.1 |
| Cervical cancer | 14 | 4 (28.6%) | 0.2-0.4 |
| Vulvar cancer | 7 | 1 | 0.5 |
| Fallopian tube cancer | 3 | 1 | 0.6 |
| Total cancer | 123 | 42 (34.1%)^{c} | |
| Benign | 86 | 9 (10.5%) | 0.2-2.8 |

^{a}p = 0.0023;
^{b}p = 0.0006;
^{c}p = 0.0002 vs. benign

### Example 3

### Detection of Hsp27-Cytochrome C Complexes

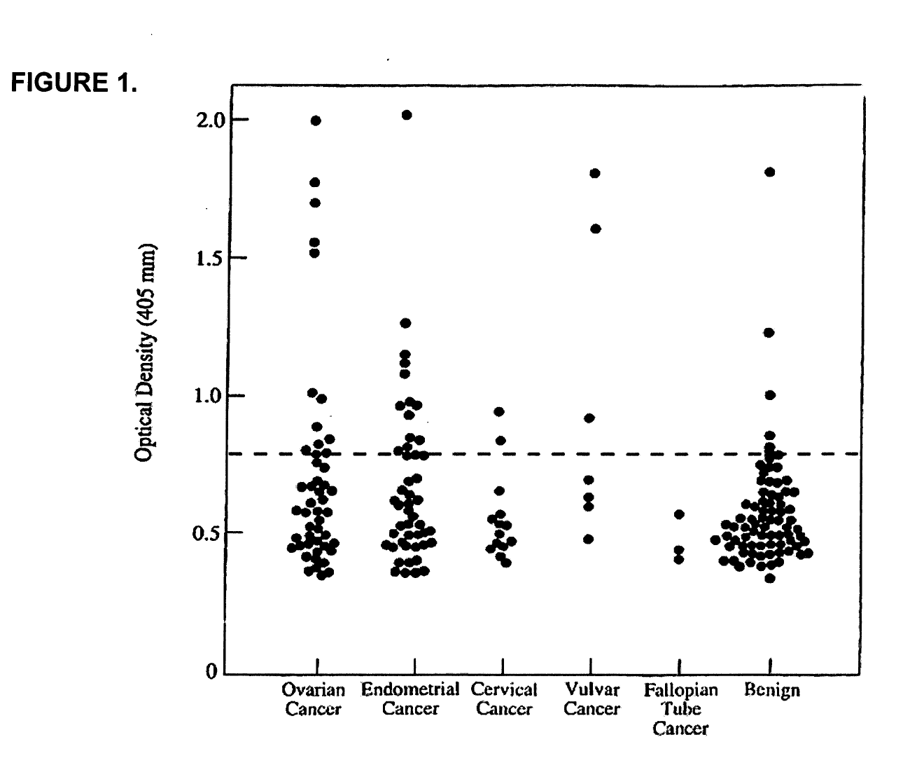

The binding of hsp27 to cytochrome C is one mechanism whereby hsp27 inhibits apoptosis and permits the survival of malignantly transformed cells. The presence of extracellular hsp27-cytochrome C complexes in cervical specimens are summarized in Table 2; individual values are shown in FIG. 1. Women with ovarian cancer or endometrial cancer had a 3-4 fold higher prevalence of hsp27-cytochrome C complexes than did women with benign conditions.

TABLE 2
Detection of hsp27-cytochrome C complexes in cervical
specimens from women with gynecologic cancers

| Diagnosis | No. women | No. hsp27-cytochrome C positive |
|---|---|---|
| Endometrial cancer | 52 | 16 (30.8%)^{a} |
| Ovarian cancer | 47 | 13 (27.7%)^{b} |
| Cervical cancer | 14 | 2 (14.3%) |
| Vulvar cancer | 7 | 3 |
| Fallopian tube cancer | 3 | 0 |
| Total cancer | 123 | 34 (27.6%)^{c} |
| Benign | 86 | 7 (8.1%) |

^{a}p = 0.008;
^{b}p = 0.004;
^{c}p = 0.0004 vs. benign

Thus, the data shows that there was a correlation between levels of hsp27 and hsp27-cytochrome C in cervical specimens (p=0.02). The percentage of ovarian cancer (44.7%) or endometrial cancer (48.1%) patients with either hsp27 or hsp27-cytochrome C in their cervical specimens also greatly exceeded their presence in the benign group (17.4%) (p<0.001).

### Example 4

### Hsp27 and Hsp27-Cytochrome C and Ovarian Cancer

The relation between detection of hsp27 or hsp27-cytochrome C complexes and ovarian cancer parameters is shown in Table 3. The percentage of patients positive in either assay was greater for women with active disease than for those whose ovarian cancer was in remission. This difference reached statistical significance, however, only for hsp27 (p<0.05). Similarly, women with stage 1-2 active ovarian cancer had a higher prevalence of hsp27 than did patients with active stage 3-4 disease (p=0.01). There was no relation between degree of tumor differentiation and detection of either hsp27 or hsp27-cytochrome C complexes.

TABLE 3
Detection of cervical hsp27 and hsp27-cytochrome
C complexes in women with active ovarian cancer

| | | No. positive | |
|---|---|---|---|
| | No. | | Hsp27- |
| Parameter | women | Hsp27 | cytochrome C |
| Active disease | 25 | 11 (44.0%)^{a} | 8 (32.0%) |
| Remission | 17 | 3 (17.6%) | 3 (17.6%) |
| Stage 1-2 active disease | 10 | 8 (80.0%)^{b} | 5 (50.0%) |
| Stage 3-4 active disease | 14 | 3 (21.4%) | 3 (21.4%) |
| Poorly differentiated | 19 | 8 (42.1%) | 6 (31.6%) |
| Moderately/well differentiated | 4 | 3 (75.0%) | 1 (25.0%) |

^{a}p < 0.05 vs. remission;
^{b}p = 0.01 vs. stage 3-4 disease

### Example 5

### Hsp27 and Hsp27-Cytochrome C and Endometrial Cancer

The relation between detection of hsp27 or hsp27-cytochrome C complexes and endometrial cancer parameters is shown in Table 4. Only 10 of the patients had active disease. With this limitation in the analysis, the results differed from women with ovarian cancer in that there were no differences between women with active disease or in remission and detection of hsp27 or hsp27-cytochrome C complexes. Only 6 women had stage 3-4 endometrial cancer, precluding analysis of the relation between stage and positivity in our assays. There was no difference in results when comparing women with stage 1 disease with those with stage 2 disease (data not shown). The endometrial cancers in 78% of our patients were moderately/well differentiated. The number of women with poorly differentiated tumors were too small for a comparative analysis.

TABLE 4
Relation between cervical hsp27 and hsp27-cytochrome
C complexes and endometrial cancer parameters

| | | No. positive | |
|---|---|---|---|
| | No. | | Hsp27- |
| Parameter | women | Hsp27 | cytochrome C |
| Active disease | 10 | 4 (40.0%) | 3 (30.0%) |
| In remission | 41 | 13 (31.7%) | 13 (31.7%) |
| Stage 1-2 disease | 44 | 17 (38.6%) | 16 (36.4%) |
| Stage 3-4 disease | 6 | 0 | 0 |
| Poorly differentiated | 11 | 3 (27.3%) | 1 (9.1%) |
| Moderately/well differentiated | 39 | 14 (35.9%) | 15 (38.5%) |

What is claimed is:

1. A method for detecting gynecological cancer in a human, the method comprising:
(i) obtaining extracellular gynecological fluid,
(ii) determining the presence of extracellular heat shock proteins (hsp) in the fluid,
wherein the presence of extracellular hsp in the fluid indicates the presence of a gynecological cancer in the human.

2. A method according to claim 1, wherein the hsp is hsp27, hsp70, hsp90 or combinations thereof.

3. A method according to claim 1, wherein the hsp is complexed to a molecule involved in apoptosis.

4. A method according to claim 3, wherein the molecule is a protein.

5. A method according to claim 3, wherein the protein is an apoptotic protein.

6. A method according to claim 5, wherein the apoptotic protein is cytochrome C, APAF-1, caspase or combinations thereof.

7. A method according to claim 1, wherein the fluid is obtained with an absorbent swab.

8. A method according to claim 1, wherein the fluid is obtained with a sponge.

9. A method according to claim 1, wherein the fluid is obtained with an aspirator.

10. A method according to claim 1, wherein the gynecological cancer is endometrial cancer, ovarian cancer, cervical cancer, vulvar cancer, fallopian tube cancer, uterine cancer or combinations thereof.

11. A method according to claim 1, wherein the presence of extracellular hsp is determined by an enzyme linked immunoabsorbant assay (ELISA).

12. A kit for detecting gynecological cancer in a human, the kit comprising an object capable of removing extracellular gynecological fluid and an antibody specific for hsp.

13. A kit according to claim 12, wherein the object is a cotton swab.

14. A kit according to claim 12, wherein the object is a sponge.

15. A kit according to claim 12, wherein the object is a aspirator.

16. A kit according to claim 15, wherein the aspirator is a syringe.

17. A kit according to claim 15, wherein the aspirator is a pipet.

18. A kit according to claim 12, wherein the kit further comprises at least one of the following: a label, buffers, standards containing hsp, materials for developing colorimetric labels, and materials for stopping colorimetric reactions.
